(19) United States
(12) Patent Application Publication
(10) Pub. No.: US 2004/0171928 A1
(43) Pub. Date: Sep. 2, 2004

(54) Method

(76) Inventors: Stefan Petersson, Malmo (SE); Sven Mansson, Malmo (SE)

Correspondence Address: Amersham Health, Inc., IP Department, 101 Camegie Center, Princeton, NJ, 08540, US

(21) Appl. No.: 10/798,023

(22) Filed: Mar. 11, 2004

Related U.S. Application Data
(63) Continuation of application No. PCT/NO02/00321, filed on Sep. 12, 2002.

(30) Foreign Application Priority Data
Sep. 12, 2001 (GB) 0122049.0

(51) Int. Cl. A61B 5/05; G01V 3/00

(52) U.S. Cl. 600/420; 324/309

(57) ABSTRACT

The present invention provides a method of magnetic resonance imaging of a sample, said method comprising:

i) administering a hyperpolarised MR imaging agent comprising non-zero nuclear spin nuclei into said sample;

ii) exposing said sample to a radiation at a frequency selected to excite nuclear spin transitions in said non-zero nuclear spin nuclei;

iii) detecting MR signals from said sample utilising spectral-spatial excitation, in combination with line scanning, point scanning and/or steady state imaging techniques; and

iv) optionally generating an image, physiological data or metabolic data from said detected signals.

### METHOD

The present invention relates to methods of magnetic resonance imaging (MRI), in particular to the study of metabolites and methods of extracting metabolic information.

In order to achieve effective contrast between MR images of different tissue types, it has long been known to administer to a subject under examination MR contrast agents (the term “MR contrast agent” in the context of the present application can be interchangeably used with the term “imaging agent”, “MR imaging agent” or “contrast agent”), e.g. paramagnetic metal species which affect relaxation times in the zones in which they are administered or at which they congregate. MR signal strength is dependent on the population difference between the nuclear spin states of the imaging nuclei. This population difference is governed by a Boltzmann distribution and is dependent on temperature and magnetic field strength.

Techniques have been developed which involve ex vivo nuclear spin polarisation of agents containing non zero nuclear spin nuclei (e.g. ³He, ¹³C, ¹⁵N,), prior to administration and MR signal measurement. The term “polarisation” in the context with the present application can be interchangeably used with the term “hyperpolarisation”. Some such techniques involve the use of polarising agents, for example conventional OMRI imaging agents or hyperpolarised gases to achieve ex vivo nuclear spin polarisation of non zero nuclear spin nuclei in an administrable MR imaging agent. By polarising agent is meant any agent suitable for performing ex vivo polarisation of an MR imaging agent.

In MRI methods involving ex vivo nuclear spin polarisation, the signal is obtained directly from the nuclei of the agent, as opposed to conventional MRI, where the signal is obtained from protons, which in turn are affected by the paramagnetic contrast agent. The hyperpolarized MR imaging agents should comprise in their molecular structure nuclei capable of emitting MR signals in a uniform magnetic field (e.g. MR imaging nuclei such as ¹³C or ¹⁵N nuclei) and capable of exhibiting a long T₁ relaxation time, and preferably additionally a long T₂ relaxation time. Such agents are referred to hereinafter as “high T₁ agents”. A high T₁ agent, a term which does not include ¹H₂O, will generally be water-soluble and have a T₁ value of at least 6 seconds in D₂O at 37 C and at a field of 7 T, preferably 8 secs or more, more preferably 10 secs or more, especially preferably 15 secs or more, more especially preferably 30 secs or more, yet more especially preferably 70 secs or more, even yet more especially preferably 100 secs or more. Unless the MR imaging nucleus is the naturally most abundant isotope, the molecules of a high T₁ agent will preferably contain the MR imaging nucleus in an amount greater than its natural isotopic abundance (i.e. the imaging agent will be “enriched” with said nuclei).

Several ways of hyperpolarising compounds comprising long T₁ nuclei, e.g. ¹³C or ¹⁵N nuclei, to produce imaging agents are known. For example, it is possible to use the ‘para-hydrogen method’—see Applicant's own earlier International Publication No. WO-A-99/24080—or dynamic nuclear polarisation (DNP)—see WO-A-99/35508, both of which are herein incorporated in their entirety.

The use of hyperpolarised MR imaging agents in MR investigations such as MR imaging has the advantage over conventional MR techniques in that the nuclear polarisation to which the MR signal strength is proportional is essentially independent of the magnetic field strength in the MR apparatus. Currently the highest obtainable field strengths in MR imaging apparatus are about 17 T, while clinical MR imaging apparatus are available with field strengths of about 0.2 to 3.0 T. Since superconducting magnets and complex magnet construction are required for large cavity high field strength magnets, these are expensive. Using a hyperpolarised imaging agent, since the field strength is less critical it is possible to make images at all field strengths from earth field (40-50 μT) up to the highest achievable fields.

Conventionally, detection of the MRI signal in MRI methods following the administration of a hyperpolarised contrast agent into a sample is via one of the standard Fourier-based methods (e.g. spin warp, EPI etc.). If the contrast agent for example comprises a compound of interest in metabolic studies, it is in this way possible to visualise the concentration of a given metabolite. In such methods, the required resolution of the image will determine the number of phase-encoding steps required. When a fast gradient echo sequence is applied, such as FLASH, the total scan time equals the number of phase-encoding steps multiplied by the repetition time. Thus, to obtain high resolution, many phase-encoding steps are required and hence the scan time will be relatively long.

When a hyperpolarised imaging agent is employed and in order to detect and visualise the changes in metabolite concentrate at two or more locations, the pulse sequence, at least when a standard Fourier transform (FT) method is used, must also collect data from areas outside of the specific “regions of interest” (ROI). The nature of the standard FT method means that it is in fact necessary to collect data from a complete ‘slice’. After the scan, the data obtained can be reconstructed into an image.

The desired spatial resolution in the ROI's will in itself dictate the number of phase-encoding steps required to sample the complete slice plane. Hence, if a high spatial resolution is required in a given ROI, a large number of phase-encoding steps will be required. This translates to a large number of excitation pulses and—as the magnetisation is divided between all the excitation pulses when using a hyperpolarised contrast agent—to a lower signal-to-noise ratio (SNR).

In the technique of chemical shift imaging, the pulse sequences used are multi-dimensional, that is at least one spatial dimension and one frequency dimension. Thus, when sampling along a slice, a strong gradient is used followed by two spatial (phase) encoding gradients. Signal collection is then performed without any gradient. In methods utilising hyperpolarised MR agents, magnetisation is divided between all the excitation pulses, thus leading to a low SNR.

In its broadest sense, the present invention relates to a method which is utilising the spectral-spatial excitation technique and which is performed after the administration of an imaging agent to a sample.

Thus viewed from one aspect the present invention provides a method of magnetic resonance imaging of a sample, preferably a human or non-human animal body (e.g. a mammalian, reptilian or avian body), said method comprising:

i) administering a hyperpolarised MR imaging agent comprising non-zero nuclear spin nuclei into said sample;

ii) exposing said sample to a radiation at a frequency selected to excite nuclear spin transitions in said non-zero nuclear spin nuclei;

iii) detecting MR signals from said sample utilising spectral-spatial excitation, in combination with line scanning, point scanning, single voxel detection and/or steady state imaging techniques, preferably in combination with steady state imaging techniques; and

iv) optionally generating an image, physiological data (e.g. pH, pO₂, pCO₂, temperature or ionic concentrations) or metabolic data from said detected signals.

If the method according to the invention is used to generate metabolic data, MR signals according to step iii) are detected after the imaging agent has left the vascular bed.

One way to alleviate the problem of low SNR as noted above is that instead of collecting a three-dimensional data set (over at least one spatial and one frequency dimension), images containing information only from specific peaks at known positions in the MR spectrum are generated. In this manner, the number of required excitations is reduced and hence the SNR is raised.

As such, the method as described above may be used to extract metabolic information. For instance, if the imaging agent comprises a hyperpolarised compound which is of interest in metabolic studies and the T₂ value of the metabolite in question is long, then the complete data collection may be performed after only one excitation of the metabolite. Hence, the SNR will be increased.

In order to collect image information from two or more metabolites, the MR spectrum must be known. The separation during the image pulse sequence is then performed using a combination of spectral and spatial selective rf excitations and standard gradient pulses. By performing the excitation using composite binomial pulses it is possible to bring one component, A say, of a two metabolite-component system, A and B say, into the xy-plane, whilst leaving the B component in the z-direction. Thus, the component of metabolite A can be separately detected. After this detection, component B can be similarly rotated into the xy-plane and detected separately.

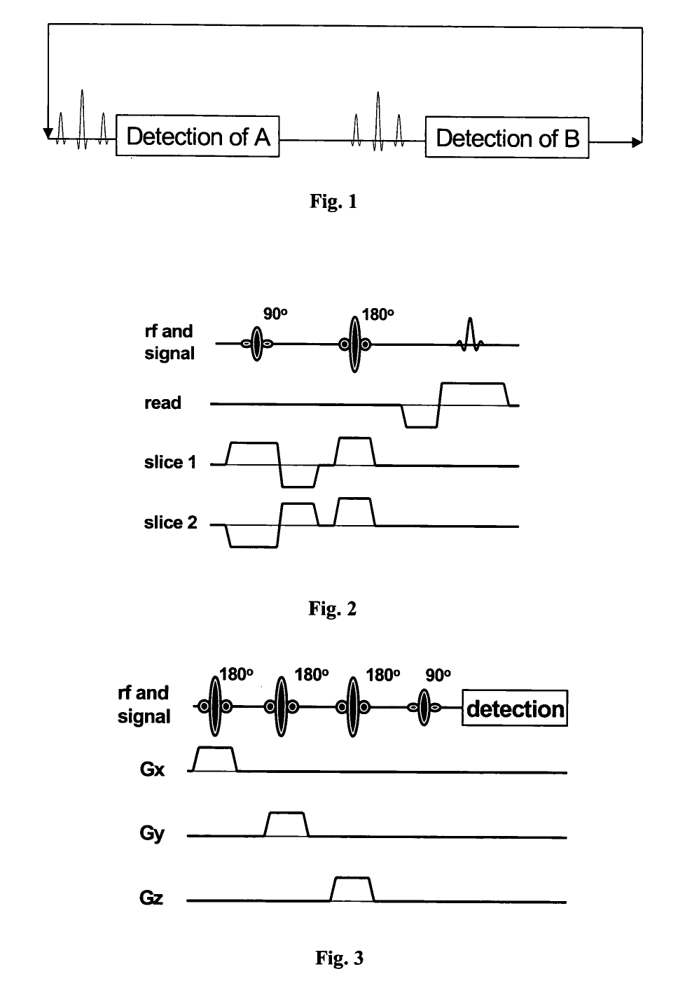

The effective T₂ relaxation time will determine whether the detection stage outlined above includes only one phase-encoding step or all the phase steps needed to reconstruct a complete image. Subsequent to the first detection interval, the peak corresponding to the second metabolite is excited using the same type of composite pulse and then the generated xy-magnetisation is detected. This sequence is shown schematically in FIG. 1 of the accompanying drawings.

If the T₂ relaxation time of the metabolites is short, then the sequence shown in FIG. 1 is repeated in order to collect all the phase-encoding steps needed to reconstruct images showing the spatial distribution of the two metabolites.

However, if the T₂ values of the metabolites are long, for example of the order of a few 100 milliseconds or more, preferably 200 milliseconds or more, more preferably 500 milliseconds or more, most preferably 1000 milliseconds or more, so-called single shot detection schemes can be employed, for example spiral or EPI gradient readout sequences. If, on the other hand, the T₂ values of the metabolites are short, for example of the order of 50 milliseconds or shorter, preferably 35 milliseconds or shorter, more preferably 20 milliseconds or shorter, most preferably 10 milliseconds or shorter, single shot detection cannot be used. Short T₂ values on this scale means that ‘new’ z-magnetisation corresponding to a specific metabolite is constantly created and thus the detection stage is carried out using several excitations.

The method of this aspect of the present invention thus makes it possible to either simultaneously or in an interleaved fashion, detect the contribution from two or more metabolites present in the same slice plane.

Preferably, the hyperpolarised MR imaging agent should comprise a compound of interest in metabolic studies.

For example, the compounds shown in the schemes below are particularly suitable. In each case, the chemical shift values of the respective ¹³C nuclei are given.

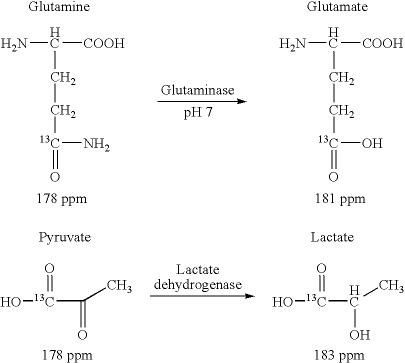

The present invention also relates in a further aspect to a method whereby MR signals are detected by line scanning (LS) whereby the above-mentioned drawbacks of lower SNR's can once again be alleviated. In this aspect, the detection step (iii) above comprises line scanning, preferably in combination with steady state imaging techniques.

When using the line scanning (LS) aspect of the invention, data from discrete lines are collected, wherein said lines include the ROI's. This has the advantages of reducing the required scan time compared to conventional FT techniques and also reduces the susceptibility of the method to both movement of the subject being imaged and blood flow. Indeed, it is found that the SNR expected from the present LS method when hyperpolarised contrast agents are used, is the same as the one delivered by a variable flip angle gradient echo (VFA-GE) sequence. In other words, the loss of SNR usually found when hyperpolarised contrast agents are used in methods incorporating conventional FT techniques is eliminated or at least reduced.

A suitable LS pulse method is shown in FIG. 2 of the accompanying drawings. It is shown in FIG. 2 that the combination of one 90 and one 180 pulse together with gradient pulses excites two tilted planes through the imaged object and thus only the MR signal from the cross-section, that is, a discrete line, will be detected.

Consequently, in this method, it is only the MR signal from the discrete line that is sampled during the sample window. Z-magnetisation outside the selected line is essentially untouched and may be detected by successive pulses. Hence, only information needed to reconstruct lines which include the ROI are collected. The number of lines required will depend on the selected resolution.

Thus, if information is only required from restricted areas, i.e. when information is required on metabolites following the administration of a contrast agent comprising a hyperpolarized compound which is of interest in a metabolic study, it is possible to significantly reduce the scan time by using the LS method herein described, rather than the standard VFA-GE method. Furthermore, this method has the advantage that it is less sensitive to movements, i.e. phase artefacts, and the method may be extended to a multi-echo version which makes it possible to obtain images with different T₂ weightings.

A further aspect of the present invention is to use so-called point scanning or single voxel detection. In this aspect, the detection step (iii) above comprises point scanning or single voxel detection, preferably in combination with steady state imaging techniques.

In this latest aspect, the spins of the nuclei in a volume element (voxel), i.e. in a ROI, are excited using a 90 pulse and then the MR signal is collected. As the volume elements under investigation can be limited to the specific ROI, the total scan time is significantly reduced. Using this method it is possible to obtain comparable SNR values for studies with hyperpolarised contrast agents as could be obtained using a standard VFA-GE sequence.

A suitable pulse sequence capable of collecting the signal from a single voxel in the manner of this aspect of the present invention is shown in FIG. 3 of the accompanying drawings. It is shown in FIG. 3 that the combination of three rf pulses together with a 90 gradient pulse excites three tilted planes through the imaged object and only the MR signal from the discrete voxel will be detected.

When the standard gradient echo (GE) or spin echo (SE) sequences are used, a high SNR is achieved using several excitations after which the MRI-signal from the complete imaged slice or volume is collected. Between the excitations the z-magnetization is partly completely restored. However, when hyperpolarized media are used this is found not to be the case. No new z-magnetization is created. Instead the z-magnetization is split due to the applied rf pulses. Previously, the variable flip angle (VFA) approach has been used. In this technique the flip angle of the excitation pulses are calculated using the expression αₙ₋₁=arctan(sin(αₙ)), where α is the flip angle (FA). If the effect due to T₁-relaxation during the sequence is ignored, all xy-magnetization components, generated after each excitation pulse, will have the same amplitude. In the case of a hyperpolarised gas (e.g. ¹²⁹Xe, ³He) the T₁ value is of the order of several seconds and thus the assumption is valid. A hyperpolarised ¹³C-contrast agent will also have very long T₁ and T₂ values. However when metabolites of said contrast agent are visualized, one has to take into account the mean life-time of the metabolite in question.

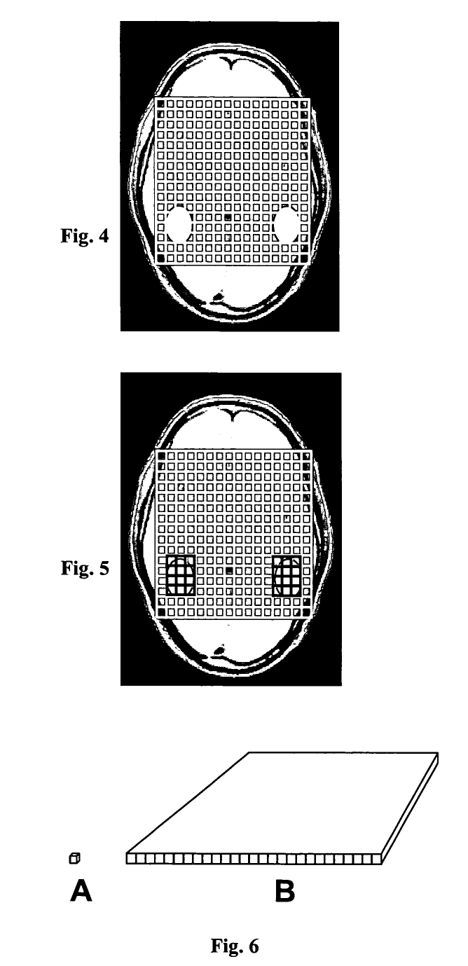

When chemical shift imaging (CSI) is performed in order to obtain ¹H-spectra the number of excitations has to at least equal the number of matrix elements. FIG. 4 of the accompanying drawings illustrates how a 16×16 matrix may be placed in order to collect the ¹H-spectrum from the ROI's. While both the x- and the y-directions are phase encoded, this method of collecting the MRI-signal will have the same effect as using an average factor of NₓN_{y} where Nₓ and N_{y} are the number of matrix elements in the x- and y-directions, respectively. Consequently, this will result in an increased SNR with a factor of 16 (equal to the square root of NₓN_{y} for a 16×16 matrix) compared to the situation if one where to collect the signal from each volume element separately using a single point scanning method. This factor is valid only if long TR is used, thus allowing the proton z-magnetization to be fully restored after each excitation. The pixel size will determine the size of the matrix size required. If this scheme were to be used in combination with hyperpolarised contrast agent, the available z-magnetization would need to be split into 256 (=16×16) excitations and thus the scan time would equal (256×TR). This splitting may be performed using VFA.

With the method according to the invention using point scanning, data can be collected from the dark ROI's indicated in FIG. 5 of the accompanying drawings only, thus, the total scan time would be reduced to (24×TR).

In addition, it is necessary to consider the effect on the SNR. A simulation system, based on a k-space partition model, has been used to evaluate the SNR in a VFA-CSI sequence compared to a single point scanning method.

The phantom objects, used to compare the expected relative SNR of the point scan (PS) method with a standard variable flip angle chemical shift image (VFA-CSI) sequence, are shown in FIG. 6 of the accompanying drawings. The volume of a given point (A in FIG. 6) extracted from the imaged sample using the PS method corresponds to the volume represented by one single element in the image matrix (B in FIG. 6) generated using the VFA-GE sequence. The results of the simulations demonstrate, that the LS- and PS methods give a comparable SNR to the VFA-CSI method, as long as an hyperpolarised imaging agent is used.

Thus, if information is only required from restricted areas, i.e. when information is required on metabolites following the injection of hyperpolarised contrast agents, it is possible to significantly reduce the scan time by using the PS method herein described compared to the scan time using the VFA-CSI approach. Furthermore, this aspect has the advantage that by reducing the scan time it becomes possible to measure local changes in the concentration of metabolites since the temporal resolution is increased. This aspect may also advantageously be used to measure the inflow of hyperpolarised contrast agents to a restricted volume, e.g. to a voxel, due to flow, diffusion or perfusion.

The final aspect of the present invention relates to methods involving steady state imaging techniques e.g. by using pulse sequences specially adapted to successfully image hyperpolarised agents with long relaxation times.

Previously, most experiments with hyperpolarised agents have focused on lung ventilation using hyperpolarised noble gases. In such experiments, rapid pulse sequences with small flip angles, e.g. FLASH are used, due to the short T₂ times of the gases in the lungs. By using hyperpolarised agents containing nuclei with extremely long relaxation times, e.g. ¹³C nuclei typically with T₁ and T₂ values greater than 10 secs, new possibilities arise in the field of physiological mapping.

When the repetition time (TR) between successive RF-excitations is short compared to the T₂ relaxation time, transverse magnetization will survive long enough to contribute to the signal collected during several successive TR intervals. This effect is referred to as “steady state” and has been thoroughly analyzed in Magn. Res. Imaging, Vol. 6 (1988), 355-368. When the signal comes from a hyperpolarised agent, a true steady state cannot be established. However, if the total duration of the imaging sequence is short compared to the T₁ relaxation time and T₂ is long compared to TR, a “pseudo steady state” (in the following, the term “steady state” is used for “pseudo steady state” also) is established. This cannot occur when imaging the lung ventilation using hyperpolarised gases (since T₂ and T₂* values are too low), but can easily be the case when utilizing a hyperpolarised agent (e.g. comprising ¹³C or ¹⁵N) in liquid phase.

When a steady state situation is reached the signal amplitude from a region where the hyperpolarised imaging agent is present will be constant and the attenuation of it will be a mix of T₁ and T₂ relaxation. If the pulse sequence used is a fully balanced gradient echo sequence (e.g. true FISP) the T₂ part of the attenuation will be a function of T₂ and not T₂*, as is common in gradient sequences. Thus, the fully balanced version of gradient sequences is the preferred choice.

The FISP and PSIF pulse sequences described in Magn. Res. Imaging, Vol. 6-(1988), 355-368 are two possible sequences for steady state imaging. However, both FISP and PSIF sequences offer poor T₂ contrast when used with small flip angles. In contrast, higher flip angles (45-90) produce a pronounced T₂ contrast, and such sequences have not been described in the literature.

The applications for the method according to the invention using T₂-contrast sensitive sequences include physiological imaging using hyperpolarised imaging agents with long relaxation times. The intrinsic T₂ relaxation rate of the agent may increase (shorter T₂) due to physiological changes (e.g. pH, temperature). If the hyperpolarised imaging agent is metabolized, the apparent T₂ relaxation rate will also increase due to the shorter half-life of the agent, thus giving reduced signal in areas with faster metabolism.

Suitable MR imaging agents for use in the methods of the present invention have been previously described by the present Applicant, for instance in WO-A-99/35508 all of which publications are herein incorporated by reference.

By “hyperpolarised” we mean polarised to a level over that found at room temperature and 1 T, preferably polarised to a polarisation degree in excess of 0.1%, more preferably in excess of 1%, even more preferably in excess of 10%.

The hyperpolarised imaging agent should preferably also exhibit a long T₂ relaxation time, preferably greater than 0.5 secs, more preferably greater than 1 sec, even more preferably greater than 5 secs.

Suitable MR imaging agents for use in the aspects of the invention may contain nuclei such as ¹H, ¹⁹F, ³Li, ¹³C, ¹⁵N, ²⁹Si, ¹²⁹Xe, ³He or ³¹P, preferably ¹³C and ¹⁵N. Most especially preferred are ¹³C nuclei.

As noted above, ¹³C and ¹⁵N are the nuclei most suited to use in the methods of the present invention with ¹³C especially preferred. ¹H nuclei have the advantages of being present in high concentration in natural abundance and having the highest sensitivity of all nuclei. ¹³C nuclei are advantageous as the background signal from hyperpolarised ¹³C nuclei is very low and much less than from, for example, ¹H nuclei. ¹⁹F nuclei have the advantage of high sensitivity. Hyperpolarisation of imaging agents comprising ³¹P nuclei allows endogenous substances to be used in all aspects of the present invention.

Where the MR imaging nucleus is other than a proton (e.g. ¹³C or ¹⁵N), there will be essentially no interference from background signals (the natural abundance of ¹³C and ¹⁵N, for instance, being negligible) and image contrast will be advantageously high. This is especially true where the MR imaging agent itself is enriched above natural abundance in the MR imaging nucleus.

The MR imaging agent should preferably be artificially enriched with nuclei (e.g. ¹⁵N and/or ¹³C nuclei) having a long T₁ relaxation time, for example more than 2 s, preferably more than 5 s, especially preferably more than 30 s.

The long T₁ relaxation time of certain ¹³C and ¹⁵N nuclei is particularly advantageous and certain MR imaging agents containing ¹³C or ¹⁵N are therefore preferred for use in the present methods. Preferably the polarised MR imaging agent has an effective nuclei ¹³C-polarisation of more than 0.1%, more preferably more than 1.0%, even more preferably more than 10%, particularly preferably more than 25%, especially more than 50% and finally most preferably more than 95%.

The MR imaging agent is more preferably ¹³C enriched at carbonyl or quaternary carbon positions, given that a ¹³C nucleus in a carbonyl group or in certain quaternary carbons may have a T₁ relaxation time typically of more than 2 s, preferably more than 5 s, especially preferably more than 30 s. Preferably the ¹³C enriched compound should be deuterium labeled, especially adjacent the ¹³C nucleus. Preferred ¹³C enriched compounds are those in which the ¹³C nuclei are surrounded by one or more non-MR active nuclei such as O, S, C or a double or triple bond.

The MR imaging agent should of course be physiologically tolerable or be capable of being provided in a physiologically tolerable, administrable form with conventional pharmaceutical or veterinary carriers or excipients. Preferred MR imaging agents are soluble in aqueous media (e.g. water).

The formulation, which preferably will be substantially isotonic, may conveniently be administered at a concentration sufficient to yield a 1 μM to 10 M concentration of the MR imaging agent in the imaging zone. However the precise concentration and dosage will of course depend upon a range of factors such as toxicity and the administration route.

Parenterally administrable forms should of course be sterile and free from physiologically unacceptable agents, and should have low osmolality to minimize irritation or other adverse effects upon administration and thus the formulation should preferably be isotonic or slightly hypertonic.

The dosages of the MR imaging agent used according to the method of the present invention will vary according to the precise nature of the MR imaging agents used and of the measuring apparatus. Preferably the dosage should be kept as low as possible while still achieving a detectable contrast effect. In general, the maximum dosage will depend on toxicity constraints.

After the polarisation, the hyperpolarised MR imaging agent may be stored at low temperature e.g. in frozen form. Generally speaking, at low temperature the polarisation is retained longer and thus polarised imaging agents may conveniently be stored e.g. in liquid nitrogen. Prior to administration, the MR imaging agent may be rapidly warmed to physiological temperatures using conventional techniques such as infrared or microwave radiation.

Embodiments of the invention are described further with reference to the following non-limiting Examples and the accompanying drawings, in which:—

FIG. 1 is an example of a pulse sequence used in the first aspect of the present invention (according to claim 1);

FIG. 2 is an outline of LS pulse sequence;

FIG. 3 is an outline of a PS pulse sequence;

FIGS. 4 and 5 illustrate how a 16×16 matrix (black grid) may be placed to collect the ¹H-spectrum from ROI's (white ellipses);

FIG. 6 shows phantom objects in the PS method;

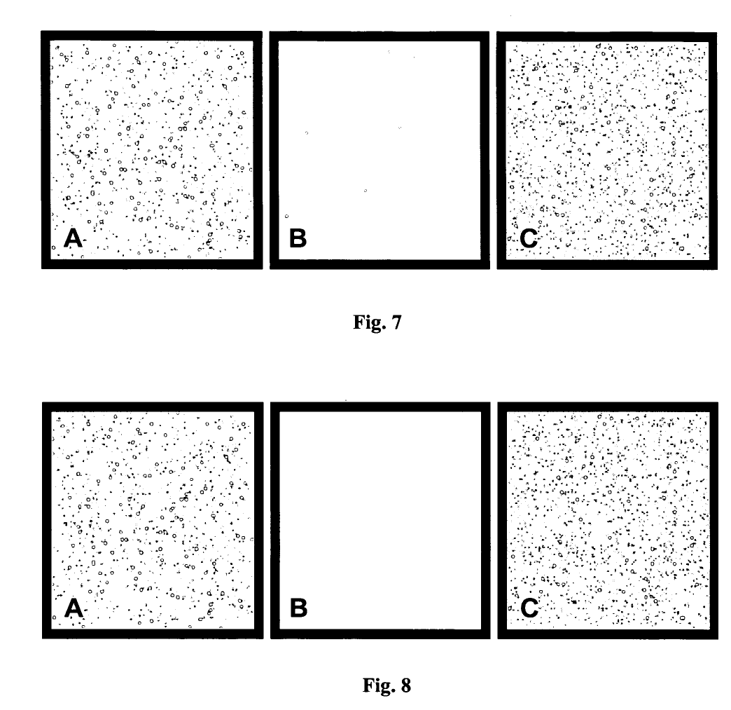

FIG. 7 shows the results from simulations using both LS and GE sequences;

FIG. 8 shows the results from simulations using both PS and CSI sequences; and

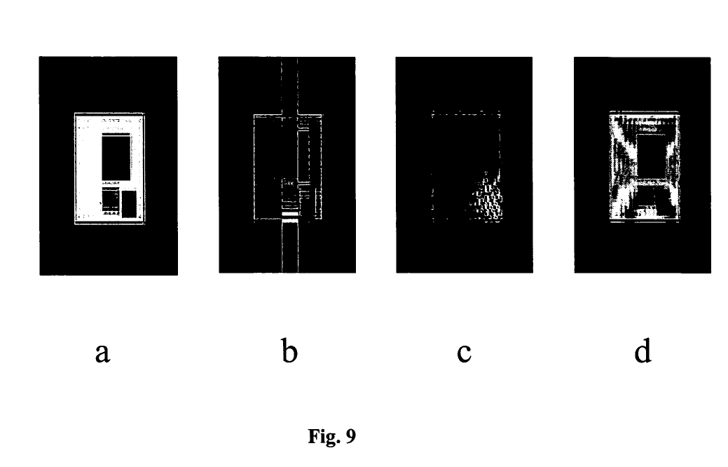

FIG. 9 shows the results of simulations of experiments with hyperpolarised agents.

### EXAMPLE 1

### Line Scanning Method

FIG. 7 of the accompanying drawings shows the results from simulations using both LS and GE sequences.

In FIG. 7a, an image generated by the LS method is shown and has a SNR of 19.4. The image in FIG. 7b is from a GE sequence with a long TR, the latter to ensure full relaxation between excitation pulses, and a flip angle of 90. In this case, the SNR is 226.5. However, the sequence leading to the image in FIG. 7b cannot be used when hyperpolarised contrast agents are used but instead the flip angle needs to be reduced to 5. The image then obtained is shown in FIG. 7c, wherein the SNR is again 19.4. Hence, the LS method produces an equivalent SNR to the GE method in the case of hyperpolarised contrast agents but the scan time is significantly reduced.

### EXAMPLE 2

### Point Scanning Method

FIG. 8 of the accompanying drawings shows the results from simulations using both PS and CSI sequences.

In FIG. 8a, an image generated by the PS method is shown and has a SNR of 17.6. The image in FIG. 8b is from a CSI sequence with a long TR, the latter to ensure full relaxation between excitation pulses, and a flip angle of 90. In this case, the SNR is 2230. However, the sequence leading to the image in FIG. 8b cannot be used when hyperpolarised media are used but instead the flip angle needs to be reduced to 0.45. The image then obtained is shown in FIG. 8c, wherein the SNR is again 17.6. Hence, the PS method produces an equivalent SNR to the CSI method in the case of hyperpolarised media.

### EXAMPLE 3

### FISP Sequence Method

FIG. 9 of the accompanying drawings shows the results of simulations of experiments with hyperpolarised imaging agents. FIG. 9a shows an image using hyperpolarised ³He gas using an FISP sequence wherein TR/TE/FA=20/3/4. The T₁ value was 36 secs, whilst T₂ was 3 ms. This is an example wherein T₂ is short and it is clear that a good SNR is obtained due to the small flip angle. FIG. 9b also shows an image using hyperpolarised ³He gas but in this case the FISP sequence has TR/TE/FA=20/3/90. Once again, the T₁ value was 36 secs and the T₂ value was 3 ms. In this case, the large flip angle causes the SNR to be low.

In FIG. 9c, ¹³C is imaged using an FISP sequence wherein TR/TE/FA=80/75/5. In this case, T₁ is 30 secs and T₂ is 30 secs in the outer region, whilst T₁ is 30 secs and T₂ is 2 secs in the inner region. This is an example wherein both T₁ and T₂ are long. With the small flip angle employed, the contrast between the two regions is poor. In FIG. 9d, ¹³C is again imaged but in this case the FISP sequence has TR/TE/FA=80/75/90. T₁ and T₂ values are as for FIG. 9c. In this case, the large flip angle ensures that the SNR is high and the T₂ contrast is significantly improved.

1. A method of magnetic resonance imaging of a sample, said method comprising:
i) administering a hyperpolarised MR imaging agent comprising non-zero nuclear spin nuclei into said sample;
ii) exposing said sample to a radiation at a frequency selected to excite nuclear spin transitions in said non-zero nuclear spin nuclei;
iii) detecting MR signals from said sample and utilising spectral-spatial excitation, in combination with line scanning, point scanning and/or steady state imaging techniques; and
iv) optionally generating an image, physiological data or metabolic data from said detected signals.

2. The method as claimed in claim 1 wherein step iii) is carried out after the agent has left the vascular bed.

3. The method as claimed in claim 1 or 2 wherein for steady state imaging a fully balanced version of gradient sequences is used.

4. The method as claimed in any of the claims 1 to 3 wherein for steady state imaging FISP or PSIF pulse sequences with high flip angles are used.

5. The method as claimed in any of the claims 1 to 4 wherein said non-zero nuclear spin nuclei are selected from the group consisting of ¹H, ³He, ³Li, ¹³C, ¹⁵N, ¹⁹F, ²⁹Si, ³¹P and ¹²⁹Xe.

6. The method as claimed in any of the claims 1 to 5 wherein said non-zero nuclear spin nuclei are selected from the group consisting of ¹³C and ¹⁵N, especially ¹³C nuclei.

7. The method as claimed in any one of the claims 1 to 6 wherein said MR imaging agent is artificially enriched with nuclei having a T₁ relaxation time of more than 5 s.

8. The method as claimed in claim 6 wherein the MR imaging agent has an effective nuclei ¹³C polarisation of more than 1%.

9. The method as claimed in claim 6 wherein the MR imaging agent is ¹³C enriched at carbonyl or quaternary carbon positions.

10. The method as claimed in claim 9 wherein said ¹³C enriched compound is deuterium labelled adjacent said ¹³C nucleus.

11. The method as claimed in any one of claims 6 to 10 wherein said ¹³C nuclei are surrounded by one or more non-MR active nuclei or entities selected from the group consisting of O, S, C or a double or triple bond.

12. The method as claimed in any of the claims 1 to 11 wherein step iii) utilises spectral-spatial excitation combined with a steady state imaging technique.

13. The method as claimed in any of the claims 1 12 wherein said imaging agent comprises a compound selected from the following: [chemical structure]
